SEQUENCE LISTING (9 sequences)

SEQ ID NO: 1 - RNA, 759 nt, synthetic construct
  source 1..759: mol_type = other RNA
atgtccgagagcttaatctacgcacctcaccccccaaatggtcgattcctagatctggggtgcggcactgggatctgggccattgatgtagcccacaagtatcccaatgctttcgttgctggagtagatctagcacctatacagcctcccaaccaccccgataactgcgagttctatgcgccttttgactttgaggcgccatggacgcttggggaaaattcttgggatctcattcatctacagatgggttgcggcagtgttctgggctggcagaatctctacaagcgaatcttaaggcatcttcagcctggggcatggtttgaacaagtggaaatagatttcgaaccccgctgcgatgatcgctccctgaatggactggcactccgggagtggtaccagtacctgaagcaggcgacacaagatacaatgcgacccatagcgcacagctcgcgggataccatcagacaccttgaggaggcaggctttacccaaatcgaccatcagatggtggggctgcctctcaacccttggcaccgtgatgaacatgagcaaaaggtagcccgttggtataacctcgcaatctctgagagtatcgagacgctcagcctcgcccctttcagtcgcatctttcactgggatctggataggatcagacagatcacagcggaggtcaagtcacaagccttcaacaaggaaatccacgcttacaatatcttacatatataccaggcacggaagccgggcggcccatcactttga

SEQ ID NO: 2 - DNA, 24 bp, synthetic construct
  source 1..24: mol_type = other DNA
gactagtcctccaaacagatggct

SEQ ID NO: 3 - DNA, 21 bp, synthetic construct
  source 1..21: mol_type = other DNA
ggtcacctcaaagtgatgggc

SEQ ID NO: 4 - DNA, 19 bp, synthetic construct
  source 1..19: mol_type = other DNA
cagctttgcccgtctgtcc

SEQ ID NO: 5 - DNA, 24 bp, synthetic construct
  source 1..24: mol_type = other DNA
aagtcaaaaggcgcatagaactcg

SEQ ID NO: 6 - DNA, 22 bp, synthetic construct
  source 1..22: mol_type = other DNA
caggctttacccaaatcgacca

SEQ ID NO: 7 - DNA, 23 bp, synthetic construct
  source 1..23: mol_type = other DNA
tttctcttaggtttacccgccaa

SEQ ID NO: 8 - DNA, 19 bp, synthetic construct
  source 1..19: mol_type = other DNA
ggtttccactatcggcgag

SEQ ID NO: 9 - DNA, 23 bp, synthetic construct
  source 1..23: mol_type = other DNA
gtctgtcgagaagtttctgatcg

(19) United States
(12) Patent Application Publication
(10) Pub. No.: US 2025/0207167 A1
(43) Pub. Date: Jun. 26, 2025

(54) ASPERGILLUS SP. L14-OE::LAEA2 AND APPLICATION THEREOF IN PRODUCING CYCLIC TRIPEPTIDES

(71) Applicant: Zhejiang University of Technology, Hangzhou (CN)

(72) Inventors: Huawei Zhang, Hangzhou (CN); Mengshi Li, Zhoushan (CN); Zhiyang Fu, Shangrao (CN); Huawei Lv, Hangzhou (CN)

(21) Appl. No.: 18/813,058

(22) Filed: Aug. 23, 2024

(30) Foreign Application Priority Data
Dec. 25, 2023 (CN) 202311793039.9

(51) Int. Cl. C12P 21/00; C12N 1/14; C12R 1/66

(52) U.S. Cl. CPC C12P 21/00; C12N 1/145; C12R 2001/66

(57) ABSTRACT

An application method includes: applying an Aspergillus sp. L14-OE::laeA2 in producing cyclic tripeptides. In the cyclic tripeptides obtained by separating and purifying a fermented product of the Aspergillus sp. L14-OE::laeA2, a yield of aspochracin is 175 mg/L, a yield of JBIR-15 is 100 mg/L, and a yield of sclerotiotide C is 25 mg/L. The yields of the compounds are significantly higher than those reported in other literature, and a separation process of obtaining the compounds is simple and economical. The cyclic tripeptides may be used to prepare drugs with antifungal activity and antioxidants activity, and for inhibiting activity of tumor cells and anti-tumor drugs. At a concentration of 10 μM, the JBIR-15 has an inhibition rate of 11.78% on a human hepatocellular carcinoma cell line (Hep-G2), and the sclerotiotide C has an inhibition rate of 21.09% on a human hepatocellular carcinoma cell line (Hep-G2).

### CROSS-REFERENCE TO RELATED APPLICATION

This application claims priority to Chinese Patent Application No. 202311793039.9, filed on Dec. 25, 2023, which is herein incorporated by reference in its entirety.

### TECHNICAL FIELD

The disclosure relates to the field of microbial pharmaceuticals, and more particularly to an engineered strain of Aspergillus sp. L14-OE::laeA2 capable of producing three cyclic tripeptides (JBIR-15, aspochracin, and sclerotiotide C) and its construction method and application.

### STATEMENT REGARDING SEQUENCE LISTING

The sequence listing associated with this application is provided in text format in lieu of a paper copy and is hereby incorporated by reference into the specification. The name of the XML file containing the sequence listing is 24082TBYX-USP1-MF-2024-0082-SL.xml. The XML file is 9,760 bytes; is created on Aug. 21, 2024; and is being submitted electronically via patent center.

### BACKGROUND

In recent years, marine fungi have become a hot research material in study of marine-derived natural products, with many biologically active compounds being discovered in their fermentation metabolites. As a member of filamentous fungi, Aspergillus niger has a rich biosynthetic gene cluster, which can control production of a large number of secondary metabolites, mainly including six structural types: pyrones, alkaloids, amides, cyclic peptides, polyketides and sterols. These compounds have important medicinal value and have huge potential applications in agriculture and medicine.

Most biosynthetic gene clusters in the filamentous fungi remain silent under laboratory culture conditions, with high potential for producing novel or high-yield compounds. In recent years, genome mining strategies (including regulation by transcription factors, promoter engineering, and heterologous expression) have been successfully developed and introduced to activate silent/low-expressed biosynthetic gene clusters for the discovery of novel or high-yield fungal secondary metabolites. The production of the secondary metabolites is controlled by a complex gene regulatory network, including global regulation. LaeA, a first global regulator in the filamentous fungi, was first discovered in Aspergillus nidulans in 2004 and has a significant coordinating effect on fungal development and secondary metabolism.

Cyclic lipopeptides are widely found in metabolic products of marine tunicates, sponges, algae, fungi, and bacteria. Structure of aspochracin-type cyclic tripeptides typically includes a unique macrocycle and a polyketide side chain. A most common characteristic of the macrocycle is composed of a twelve-membered ring (composed of alanine-valine-ornithine) or a thirteen-membered ring (composed of alanine-valine-lysine). Only 15 types of the aspochracin-type cyclic tripeptides are obtained from natural sources, and their structures mainly differ in the polyketide side chain, amino acid composition and the level of N-methylation of amino acids, exhibiting different antifungal, anti-inflammatory, and antioxidant activities. According to literature searches, two major characteristics of the aspochracin are extremely low mammalian toxicity and recognized insecticidal activity (against silkworms and American moths). The metabolite aspochracin found in Aspergillus ochraceus has the highest potency (64 milligrams per liter, mg/L). The highest potencies of JBIR-15 found in Aspergillus sclerotiorum Sp080903f04 and sclerotiotide C found in Aspergillus sclerotiorum PT06-1 are 1.48 mg/L and 0.13 mg/L, respectively. The JBIR-15 reported has anti-Candida albicans activity [minimum inhibitory concentration (MIC)=30 micrograms per milliliter (μg/mL)]. However, in biosynthesis methods in the related art, yields are all relatively low.

There currently are no reports of strains that produce high yields of the JBIR-15, the aspochracin, and the sclerotiotide C cyclic lipopeptide compounds, and there is also a lack of research on their biological activities. The global regulatory factor mining strategy may be an effective approach to uncover silent unknown biosynthetic clusters in fungi and to increase the yield of active small molecule compounds.

### SUMMARY

A purpose of the disclosure is to provide an Aspergillus sp. L14-OE::laeA2 capable of producing cyclic tripeptides (JBIR-15, aspochracin, and sclerotiotide C) with high yields and its application, which solves problems that yields of the cyclic tripeptides are low and discovers new applications of the cyclic tripeptides in fields of anti-tumor and antioxidant drugs. The Aspergillus sp. L14-OE::laeA2 is expected to be used as a drug lead in industrial production. Technical solutions of the disclosure are below.

The disclosure provides the Aspergillus sp. L14-OE::laeA2 capable of producing cyclic tripeptides with high yields. The Aspergillus sp. L14-OE::laeA2 is preserved at China Center for Type Culture Collection (CCTCC), a preservation number is CCTCC NO: M20232057, a preservation date is Oct. 27, 2023, and a preservation address is Wuhan University, Wuhan, China, zip code is 430072.

In the disclosure, a fragment gene of a global regulatory factor LaeA is overexpressed in a genome of a wild-type strain Aspergillus niger L14 (collected from the symbiotic Aspergillus niger within a marine sponge Reniera in Xinghai bay, Dalian city, Liaoning province, China) to obtain an engineered strain (the Aspergillus sp. L14-OE::laeA2) capable of producing the cyclic tripeptides with high yields. The cyclic tripeptides include the JBIR-15, the aspochracin, and the sclerotiotide C.

Specific steps of a construction method of the Aspergillus sp. L14-OE::laeA2 are as follows:
(1) obtaining the fragment gene of the global regulatory factor LaeA from the Aspergillus niger L14 by using polymerase chain reaction (PCR) and double enzyme digestion techniques, and constructing a recombinant plasmid pCAMBIA-1301:LaeA containing the fragment gene; where the nucleotide sequence of the global regulatory factor LaeA is shown as SEQ ID NO: 1;
(2) introducing the recombinant plasmid obtained in the step (1) into Escherichia coli (E. coli) DH5α competent cells, picking transformants and then shaking the transformants overnight to amplify and extract the recombinant plasmid to obtain an amplified plasmid;
(3) introducing the amplified plasmid obtained in the step (2) into Agrobacterium tumefaciens AGL-1 by a freeze-thaw method to obtain a transformed plasmid, and then mini-prepping the transformed plasmid to obtain a mini-prepped plasmid; and
(4) transferring the mini-prepped plasmid in the step (3) into the genome of the Aspergillus niger L14 by Agrobacterium-mediated fungal transformation method under the induction of acetosyringone to obtain the engineered strain Aspergillus sp. L14-OE::laeA2 capable of producing the JBIR-15, the aspochracin, and the sclerotiotide C with high yields.

The disclosure further provides an application method including: applying the Aspergillus sp. L14-OE::laeA2 in producing the cyclic tripeptides. The application method includes the following steps: fermenting and culturing the Aspergillus sp. L14-OE::laeA2 to obtain fermentation broth, and separating and purifying the fermentation broth to obtain the cyclic tripeptide compounds. The cyclic tripeptides include compound (I, aspochracin), compound (II, JBIR-15) and compound (III, sclerotiotide C) presented as follows:

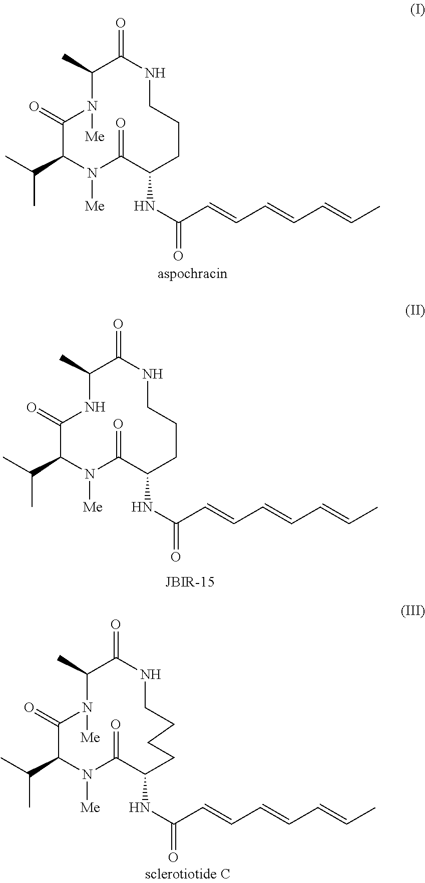

The fermenting and culturing the Aspergillus sp. L14-OE::laeA2 includes following steps:
(1) inoculating the Aspergillus sp. L14-OE::laeA2 into a potato dextrose agar (PDA) medium and then activating the Aspergillus sp. L14-OE::laeA2 at 30° C. for 3-4 days to obtain activated Aspergillus, inoculating the activated Aspergillus into a potato dextrose broth (PDB) medium and then culturing the activated Aspergillus at 30° C. and 180-200 revolutions per minute (rpm) for 3 days to obtain seed liquid (i.e., seed culture); and
(2) inoculating the seed liquid in the step (1) into a Czapek medium at a volume concentration in a range of 1-5% (3% in an embodiment) followed by culturing at 30° C. and 180-200 rpm for 15 days to obtain the fermentation broth.

In an embodiment, the PDA medium includes 20 grams per liter (g/L) of glucose, 200 g/L of potatoes (Solanum tuberosum), and 15-18 g/L of agar, with distilled water as a solvent and potential of hydrogen (pH) of natural (i.e., an unadjusted pH, which is about 7). The PDB medium includes 200 g/L of potatoes and 20 g/L of glucose with distilled water as a solvent.

In an embodiment, the Czapek medium includes 30 g/L of sucrose, 3 g/L of sodium nitrate (NaNO₃), 0.5 g/L of magnesium sulfate heptahydrate (MgSO₄·7H₂O), 0.5 g/L of potassium chloride (KCl), 0.01 g/L of ferrous sulfate (FeSO₄) and 1 g/L of dipotassium hydrogenphosphate (K₂HPO₄) with water as solvent and pH of natural.

The separating and purifying the fermentation broth includes:
filtering the fermentation broth through eight-layer gauze to separate fungal liquid and mycelium, adding the fungal liquid with an equal volume of ethyl acetate followed by extracting to obtain an upper layer (in an embodiment, extracting twice to obtain two upper layers, and combining the two upper layers to obtain the upper layer), and concentrating the upper layer to dryness by rotary evaporation under reduced pressure to obtain a crude fermentation extract; and dissolving the crude fermentation extract with methanol alcohol followed by diluting with the methanol alcohol to obtain a diluted solution, filtering the diluted solution with a 0.22 micrometers (μm) organic filter to obtain a filtrate, performing isocratic elution on the filtrate by an analytical column of high-performance liquid chromatography (HPLC) with a mobile phase of acetonitrile:water at a volume ratio of 35:65 to obtain effluents from 6^{th} minute (min) to 7^{th} min, 8^{th} min to 9^{th} min and 10.8^{th} min to 11.2^{nd} min respectively, and removing solvents from the effluents by rotary evaporation followed by drying at 25° C. to obtain the compound (I), the compound (II) and the compound (III) respectively.

In an embodiment, HPLC conditions include: an ultraviolet-visible (UV-VIS) liquid chromatography instrument, a detector: Shimadzu® SPD-M40, and a HPLC pump: Shimadzu® LC-20AT; the analytical column is a C18 column of 4.6×250 millimeters (mm), a flow rate is 1.0 milliliters per minute (mL/min), a column temperature is 40° C., a detection wavelength is 210 nanometers (nm) and an injection volume is 10 microliters (μL).

The disclosure further provides an application method including: applying the cyclic tripeptides prepared by the Aspergillus sp. L14-OE::laeA2 in preparing antioxidants drugs.

The disclosure further provides an application method including: applying the cyclic tripeptides prepared by the Aspergillus sp. L14-OE::laeA2 in preparing drugs to inhibit activity of tumor cells. The tumor cells include: a human brain glioma cell line (HEB) and a human hepatocellular carcinoma cell line (Hep-G2).

Compared to the related art, the disclosure has below beneficial effects: the disclosure constructs the engineered strain capable of producing the JBIR-15, the aspochracin and the sclerotiotide C with high yields. In the cyclic tripeptides obtained by separating and purifying the fermentation broth, a yield of the aspochracin is 175 milligrams per liter (mg/L), a yield of the JBIR-15 is 100 mg/L, and a yield of the sclerotiotide C is 25 mg/L. The yields of the compounds are significantly higher than those reported in other literature, and a separation process of obtaining the compounds is simple and economical.

The cyclic tripeptides obtained by the disclosure may be used to prepare drugs with antifungal activity, such as the JBIR-15, which has a MIC value of 32 μg/mL against Candida albicans.

The cyclic tripeptides obtained by the disclosure may be used to prepare drugs with antioxidants activity. The JBIR-15, the aspochracin and the sclerotiotide C all have moderate antioxidant activity.

The cyclic tripeptides obtained by the disclosure may be used to prepare the drugs for inhibiting the activity of the tumor cells and anti-tumor drugs. The tumor cells include the human brain glioma cell line (HEB) and the human hepatocellular carcinoma cell line (Hep-G2). At a concentration of 10 micromoles (μM), the JBIR-15 has an inhibition rate of 11.78% on the human hepatocellular carcinoma cell line (Hep-G2), and the sclerotiotide C has an inhibition rate of 21.09% on the human hepatocellular carcinoma cell line (Hep-G2).

### BRIEF DESCRIPTION OF DRAWINGS

FIG. 1 illustrates a gel electrophoresis diagram of PCR amplification products after enzyme digestion and purification in Step 1 of an Embodiment 1; Lane M: DNA marker; Lane 1: PCR products after enzyme digestion and purification.

FIG. 2 illustrates a gel electrophoresis diagram of a plasmid pCAMBIA1303-TrpC-Hygro-gpdA-GFP after restriction endonuclease digestion in Step 2 of the Embodiment 1; Lane M: DNA marker, Lanes 1-4: Products after double enzyme digestion of the plasmid pCAMBIA1303-TrpC-Hygro-gpdA-GFP.

FIG. 3 illustrates a validation PCR gel image of an overexpression plasmid pCAMBIA-1301:LaeA; Lane M: DNA marker; Lane 1: a PCR detection gel image using verify1-F/R as forward and reverse primers to verify an upstream junction of a recombinant plasmid; Lane 2: a PCR detection gel image using verify2-F/R as forward and reverse primers to verify a downstream junction of the recombinant plasmid.

FIG. 4 illustrates a validation PCR gel image of the plasmid pCAMBIA-1303:LaeA before and after transformation into Agrobacterium tumefaciens AGL-1; Lane M: DNA marker; Lane 1: Original strain Agrobacterium tumefaciens AGL-1; Lanes 2 and Lane 3: Agrobacterium tumefaciens with successfully introduced plasmid.

FIG. 5 illustrates a expression level diagram of a LaeA gene in an engineered strain L14-OE::laeA2 compared to a wild-type strain L14, as detected by quantitative PCR (qPCR).

FIG. 6 illustrates a schematic High-performance liquid chromatography (HPLC) analysis diagram of fermented products of the strain L14-OE::laeA2; (A) represents a detection spectrum of a fermentation product of the wild-type strain L14; (B) represents a detection spectrum of a fermentation product of the engineered strain L14-OE::LaeA1.

FIG. 7 illustrates a negative ion electrospray ionization-mass spectrometry (ESI-MS) spectrum of cyclic tripeptide aspochracin.

FIG. 8 illustrates a positive ion ESI-MS spectrum of the cyclic tripeptide aspochracin.

FIG. 9 illustrates a negative ion ESI-MS spectrum of cyclic tripeptide JBIR-15.

FIG. 10 illustrates a positive ion ESI-MS spectrum of the cyclic tripeptide JBIR-15.

FIG. 11 illustrates a negative ion ESI-MS spectrum of cyclic tripeptide sclerotiotide C.

FIG. 12 illustrates a positive ion ESI-MS spectrum of the cyclic tripeptide sclerotiotide C.

FIG. 13 illustrates a proton nuclear magnetic resonance (¹H NMR) spectrum (in CDCl₃) of the cyclic tripeptide aspochracin.

FIG. 14 illustrates a carbon-13 nuclear magnetic resonance (¹³C NMR) spectrum (in CDCl₃) of the cyclic tripeptide aspochracin.

FIG. 15 illustrates a ¹H NMR spectrum (in CDCl₃) of the cyclic tripeptide JBIR-15.

FIG. 16 illustrates a 13C NMR spectrum (in CDCl₃) of the cyclic tripeptide JBIR-15.

FIG. 17 illustrates a 1H detected heteronuclear multiple bond correlation (HMBC) diagram (in CDCl₃) of the cyclic tripeptide JBIR-15.

FIG. 18 illustrates a ¹H NMR spectrum (in CDCl₃) of the cyclic tripeptide sclerotiotide C.

### DETAILED DESCRIPTION OF EMBODIMENTS

The disclosure is further described below through specific embodiments. However, the protection scope of the disclosure is not limited to this.

Culture media used in the embodiments are below.

(1) A PDB medium: 200 g/L of potatoes and 20 g/L of glucose with distilled water as a solvent and pH of natural.

(2) A PDA medium, obtained by adding agar to the PDB medium.

(3) A fermentation medium (Czapek medium): 30 g/L of sucrose, 3 g/L of NaNO₃, 0.5 g/L of Na₂SO₄·7H₂O, 0.5 g/L of KCl, 0.01 g/L of FeSO₄ and 1 g/L of K₂HPO₄ with water as solvent and pH of natural.

(4) An induction liquid medium (IM liquid medium): 2.05 g/L of K₂HPO₄, 1.45 g/L of monopotassium dihydrogen phosphate (KH₂PO₄), 0.5 g/L of ammonium sulfate ((NH₄)₂SO₄), 0.5 g/L of MgSO₄·7H₂O, 0.15 g/L of sodium chloride (NaCl), 0.066 g/L of calcium chloride (CaCl₂)), 0.00248 g/L of ferrous sulfate heptahydrate (FeSO₄·7H₂O), 1.8 g/L of glucose and 5 milliliters per liter (mL/L) of glycerol are sterilized with moist heat at 121° C. for 20 min, and then cooled to 50° C. to obtain a cooled solution, the cooled solution is added with 40 mL/L of 1 mol/L 2-(N-morpholino) ethanesulfonic acid aqueous solution and 1 mL/L of 200 millimoles per liter (mmol/L) acetosyringone solution [acetosyringone (C₁₀H₁₂O₄) is dissolved with dimethyl sulfoxide (DMSO), and then sterilized by filtration through a 0.22 μm organic membrane filter] to obtain the IM liquid medium.

(5) An IM solid medium, obtained by adding 15 g/L of agar to the IM liquid medium.

(6) A Luria-Bertan (LB) liquid medium: 10.0 g/L of tryptone, 5.0 g/L of yeast extract and 1.0 g/L of NaCl, with water as a solvent and pH of natural.

(7) A LB solid medium, obtained by adding 20.0 g/L of agar powder to the LB liquid medium.

(8) A yeast extract beef (YEB) liquid medium: 5 g/L of beef extract, 1 g/L of yeast extract, 5 g/L of peptone, 5 g/L of sucrose and 5 g/L of MgSO₄·7H₂O, with water as a solvent and pH of natural.

(9) A YEB solid medium, obtained by adding 15 g/L of agar to the YEB liquid medium.

(10) A Sabouraud liquid medium: 10 g/L of peptone and 40 g/L of glucose, with water as a solvent and pH of natural.

(11) A Sabouraud solid medium, obtained by adding 15 g/L of agar to the Sabouraud liquid medium.

A parental strain (also referred to as original strain) used in the embodiments of the disclosure is Aspergillus niger L14, which is disclosed at “Genomic and AntiSMASH Analyses of Marine-Sponge-Derived Strain Aspergillus niger L14 Unveiling Its Vast Potential of Secondary Metabolites Biosynthesis. Journal of Fungi, 2022, 8 (6): 591”.

### Embodiment 1: Construction of an Engineered Strain Aspergillus sp. L14-OE::laeA2

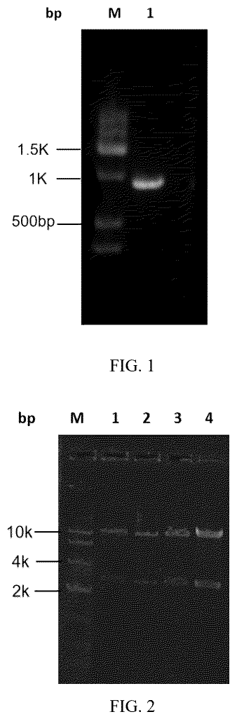

1. Extracting LaeA gene from the Aspergillus niger: total ribonucleic acid (RNA) is extracted from the Aspergillus niger L14 using a filamentous fungal RNA extraction kit; a first strand of complementary deoxy ribonucleic acid (cDNA) is synthesized from the total RNA by reverse transcription-PCR (RT-PCR) by using a reaction system as shown in Table 1. The first strand of the cDNA is amplified by PCR with primers laea-F/R to obtain a LaeA target gene fragment (a nucleotide sequence is shown as SEQ ID NO: 1). Subsequently, the LaeA target gene fragment is purified and then recovered to obtain a first recovered product, and the first recovered product is verified using agarose gel electrophoresis. Results are shown in FIG. 1.

Primer laea-F:
(SEQ ID NO: 2)
GACTAGTCCTCCAAACAGATGGCT.
Primer laea-R:
(SEQ ID NO: 3)
GGTCACCTCAAAGTGATGGGC.

TABLE 1
the reaction system for synthesizing the first strand of the cDNA

| | Total RNA less | |
|---|---|---|
| RNA template | than 1 μg | |
| 2 × StarScript III Butter (with primer) | 10 | μL |
| StarScript III RT Enzyme Mix | 1 | μL |

| | |
|---|---|
| Nuclease-free water (DEPC-treated) | Make up to 20 μL |

2. Constructing an Overexpression Plasmid pCAMBIA-1301:LaeA

A binary overexpression plasmid pCAMBIA1303-TrpC-Hygro-gpdA-GFP (purchased from Wuhan Miaoling Biology Inc.) is digested with SpeI and BstEII restriction endonucleases according to a system in Table 2, a 10199 base pairs (bp) gene fragment as a second recovered product (i.e., digested plasmid large fragment) is recovered. A gel electrophoresis image of the 10199 bp gene fragment after double enzyme digestion is shown in FIG. 2.

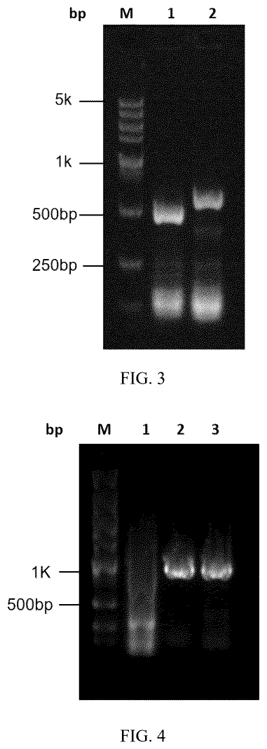

The LaeA target gene fragment is digested with the SpeI and BstEII restriction endonucleases and then recovered to obtain a third recovered product (i.e., digested LaeA gene). A concentration ratio of the second recovered product and the third recovered product is calculated, and the second recovered product and the third recovered product are connected overnight by T4 ligase in a reaction system of Table 3 to obtain a recombinant plasmid pCAMBIA-1301:LaeA of the overexpression LaeA gene (i.e., the overexpression plasmid pCAMBIA-1301:LaeA). The recombinant plasmid pCAMBIA-1301:LaeA is verified by primers verify-F/R, with results shown in FIG. 3.

verify1-F:
(SEQ ID NO: 4)
CAGCTTTGCCCGTCTGTCC.
verify1-R:
(SEQ ID NO: 5)
AAGTCAAAAGGCGCATAGAACTCG.
verify2-F:
(SEQ ID NO: 6)
CAGGCTTTACCCAAATCGACCA.
verify2-R:
(SEQ ID NO: 7)
TTTCTCTTAGGTTTACCCGCCAA.

TABLE 2
a double-enzyme digestion reaction
system for plasmid and PCR products

| | | |
|---|---|---|
| Component | Plasmid DNA | PCR Product |

| | | |
|---|---|---|
| DNA | 2 μL (≤1 μg) | 5 μL (≤0.2 μg) |

| | | | | |
|---|---|---|---|---|
| MinuteCutTM BstE II | 1 | μL | 1 | μL |
| MinuteCutTM SpeI | 1 | μL | 1 | μL |
| 10 × MinuteCutTM Buffer | 2 | μL | 3 | μL |
| ddH₂O | 14 | μL | 20 | μL |
| Total | 20 | μL | 30 | μL |

TABLE 3
the reaction system for T4 DNA ligase

| Component | System (μL) | |
|---|---|---|
| ddH₂O | To 10 μL | |
| 10 × Ligase Buffer | 1 μL | |
| digested LaeA gene | 10 μL | (0.3 picomoles, pmol) |
| digested plasmid large fragment | 7 μL | (0.03 pmol) |

| | |
|---|---|
| T4 DNA Ligase | 1 μL |
| (400 units per microliter, U/μL) | |

3. Constructing the Engineered Strain Aspergillus sp. L14-OE::laeA2

The overexpression plasmid pCAMBIA-1301:LaeA is amplified in Escherichia coli (E. coli) DH5α competent cells and then grown at 37° C. on the LB solid medium containing 50 μg/mL of kanamycin to obtain a growth plasmid. Transformants are picked from the growth plasmid and then shaken overnight at the LB liquid medium at 37° C. and 180 rpm followed by mini-prepping plasmid to obtain a mini-prepped plasmid. The mini-prepped plasmid is introduced into Agrobacterium tumefaciens AGL-1 by a freeze-thaw method to obtain plasmid-introduced Agrobacterium tumefaciens AGL-1. The Agrobacterium tumefaciens AGL-1 is activated and amplified on the YEB solid medium and the YEB liquid medium containing 25 μg/mL of rifampicin. The plasmid-introduced Agrobacterium tumefaciens AGL-1 is verified by PCR with primers Hyg-F/R and a system in Table 4. Verification results are shown in FIG. 4.

Hyg-F:
(SEQ ID NO: 8)
GGTTTCCACTATCGGCGAG.
Hyg-R:
(SEQ ID NO: 9)
GTCTGTCGAGAAGTTTCTGATCG.

TABLE 4
the system for PCR amplification

| Component | System (uL) |
|---|---|
| ddH₂O | up to 50 μL |
| 2 × Phanta Flash Master | 25 |
| Mix (Dye Plus) | |
| Verify -F (10 μM) | 2 |
| Verify-R (10 μM) | 2 |
| Template | 2 |
| Total volume | 50 |

4. The plasmid-introduced Agrobacterium tumefaciens AGL-1 is pre-induced by the IM liquid medium containing 200 μM of acetosyringone, and then shaken at 28° C. and 180 rpm for 5 hours until optical density at 600 nm (OD₆₀₀)=0.5 to obtain pre-induced Agrobacterium tumefaciens AGL-1. The pre-induced Agrobacterium tumefaciens AGL-1 is mixed with a fresh original strain Aspergillus niger L14 at a concentration of 1×10⁷ colony forming units per milliliter (CFU/mL) and then spread at the IM solid medium containing 200 μM of the acetosyringone and 25 μg/mL of the rifampicin, then, the IM solid medium is placed in an incubator at 24° C. and wrapped in aluminum foil for co-culture in the dark for 48 hours to perform transformation of Aspergillus niger mediated by Agrobacterium tumefaciens with a nitrocellulose membrane as a transfer membrane. After 48 hours, the transfer membrane is spread at the PDA medium containing 250 μg/mL of hygromycin and 200 μg/mL of cefotaxime sodium and then cultured in an incubator at 28° C. for 3-7 days to obtain mutant strains.

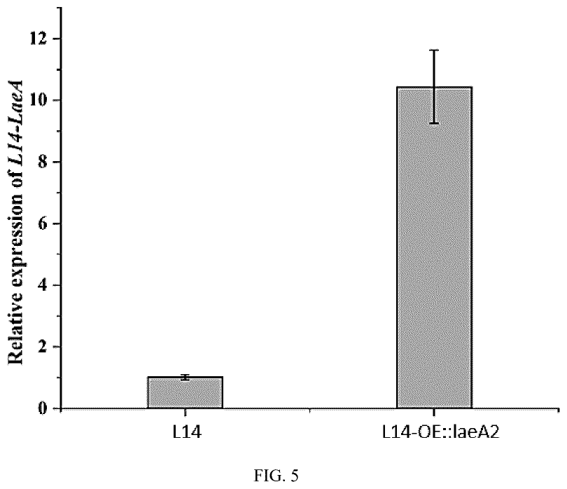

A stable mutant strain is selected from the mutant strains, a LaeA gene expression level of the stable mutant strain is measured by fluorescence quantitative PCR (qPCR) according to a system in Table 5. Results are shown in FIG. 5. Compared to the wild-type Aspergillus niger L14, the LaeA gene of the stable mutant strain is nearly ten times overexpressed. The stable mutant strain is the engineered strain Aspergillus sp. L14-OE::laeA2.

TABLE 5
qPCR reaction system

| Component | System (μL) | |
|---|---|---|
| 2 × SYBR real-time | 10 | μL |
| PCR premixture | | |
| laea-F | 0.4 | μL |
| laea-R | 0.4 | μL |

| | |
|---|---|
| cDNA | 1 |
| RNase free dH₂O | Up to 20 μL |

### Embodiment 2: Fermentation of the Engineered Strain Aspergillus sp. L14-OE::laeA2

(1) Activated culture: the engineered strain Aspergillus sp. L14-OE::laeA2 is inoculated into the slant PDA medium and then cultured in an incubator at 30° C. for 3-4 days to obtain activated Aspergillus.

(2) Seed culture: a loopful of the activated Aspergillus is inoculated into the PDB medium and then shaken at 200 rpm at 30° C. for 3 days to obtain seed liquid.

(3) Fermentation culture: the seed liquid obtained in the step (2) is inoculated into 25 L of the Czapek medium at an inoculation volume of 30 mL/L, and then shaken at 200 rpm at 30° C. for 15 days to obtain a fermented mixture.

### Embodiment 3: Extraction, Separation and Identification of Compounds

### 1. The Extraction and Separation of the Compounds

30 L of the fermented mixture prepared in the embodiment 2 is filtered through eight-layer gauze to separate fungal liquid and mycelium. The fungal liquid is taken and added with an equal volume of ethyl acetate followed by extracting twice to obtain two upper layers, the two upper layers are combined to obtain an upper layer. The upper layer is concentrated to dryness by rotary evaporation under reduced pressure to obtain a crude fermentation extract paste.

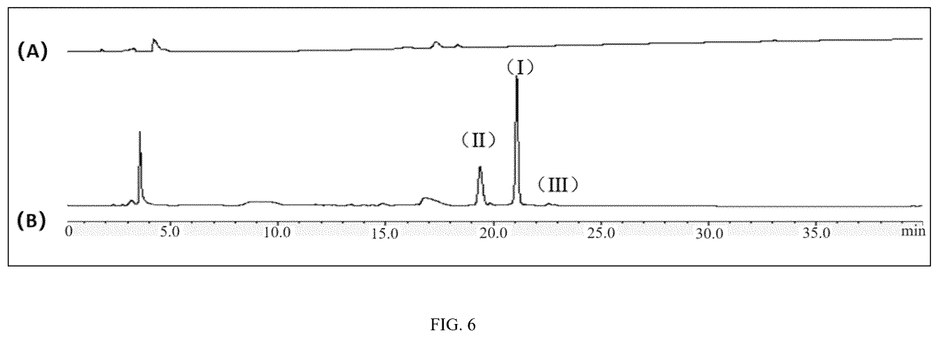

The crude fermentation extract paste is dissolved with 12 mL of methanol alcohol (also referred to as chromatographic methanol) to obtain an extract solution, 20 μL of the extract solution is diluted with methanol alcohol to obtain a diluted solution, the diluted solution is filtered through a 0.22 μm organic filter to obtain a filtrate, the filtrate is detected by HPLC, with the result shown as B in FIG. 6. Fermentation liquid of the original Aspergillus niger L14 is prepared under the same conditions and detected by the HPLC, with the result shown as A in FIG. 6.

HPLC conditions include: a UV-VIS liquid chromatography instrument, a detector: Shimadzu® SPD-M40, and a HPLC pump: Shimadzu® LC-20AT; the analytical column is a C18 column of 4.6×250 mm, a flow rate is 1.0 ml/min, a column temperature is 40° C., a detection wavelength is 210 nm and an injection volume is 10 μL. A mobile phase of gradient elution is from 10% to 100% acetonitrile.

20 μL of the extract solution is taken and filtered by the 0.22 μm organic filter to obtain a filtrate, the filtrate is then prepared by HPLC. The isocratic elution is performed on the filtrate by the analytical column of the HPLC with the mobile phase of acetonitrile:water at a volume ratio of 35:65 to obtain effluents from 6^{th} min to 7^{th} min, 8^{th} min to 9^{th} min and 10.8^{th} min to 11.2^{nd} min respectively, solvents are removed from the effluents by rotary evaporation followed by drying at 25° C. to obtain 8.75 mg of a compound (I), 5.00 mg of a compound (II) and 1.25 mg of a compound (III) respectively. A yield of the compound (I) is 175 mg/L, a yield of the compound (II) is 100 mg/L, and a yield of the compound (III) is 25 mg/L.

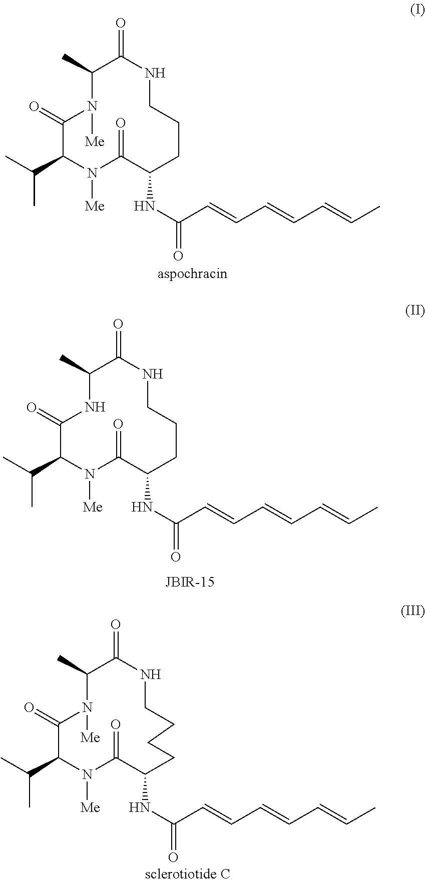

### 2. Structural Identification of the Compounds

### (1) Physical Properties

Compound I: yellow oily substance, soluble in methanol and dichloromethane.

Compound II: yellow oily substance, soluble in methanol and dichloromethane.

Compound III: yellow oily substance, soluble in methanol and dichloromethane.

### (2) Structure Elucidation

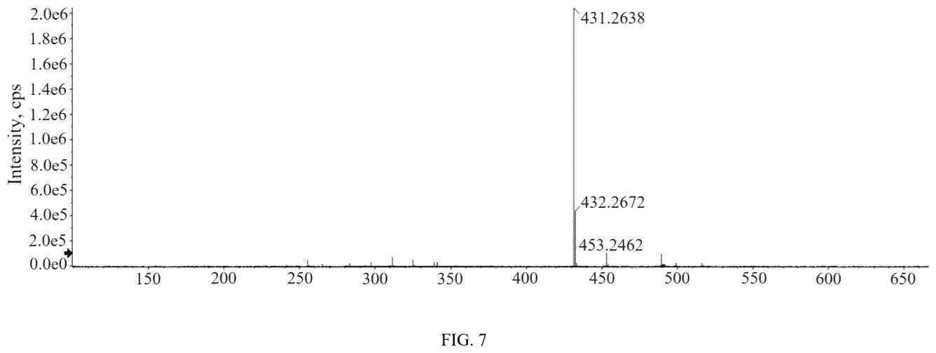

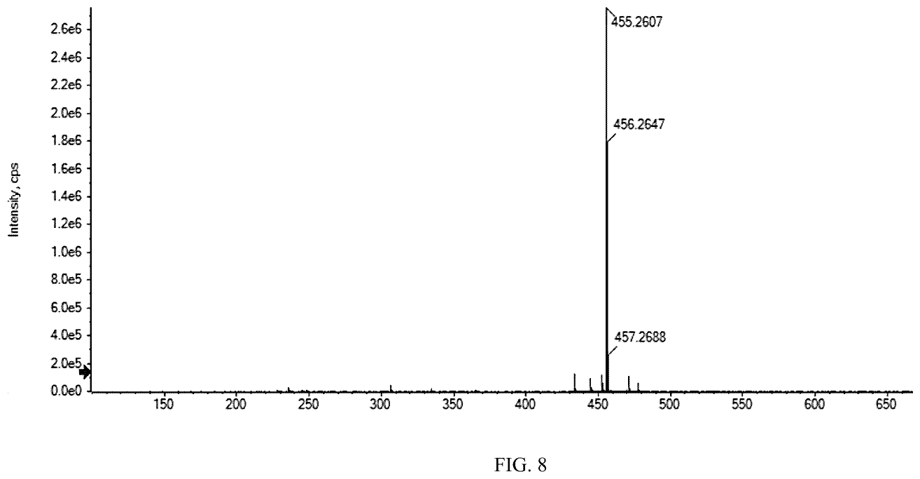

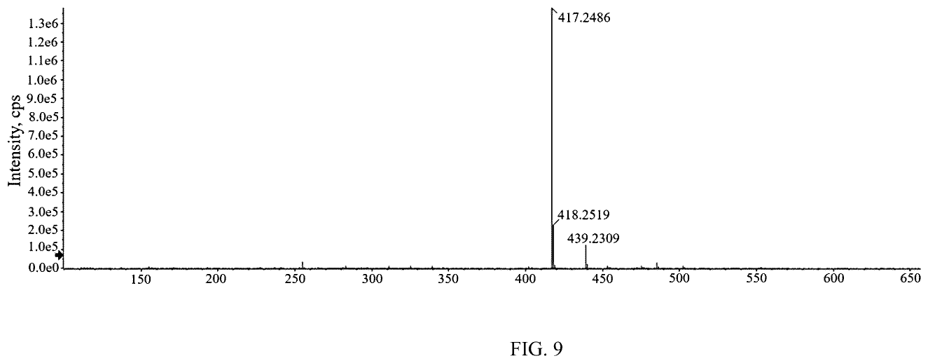

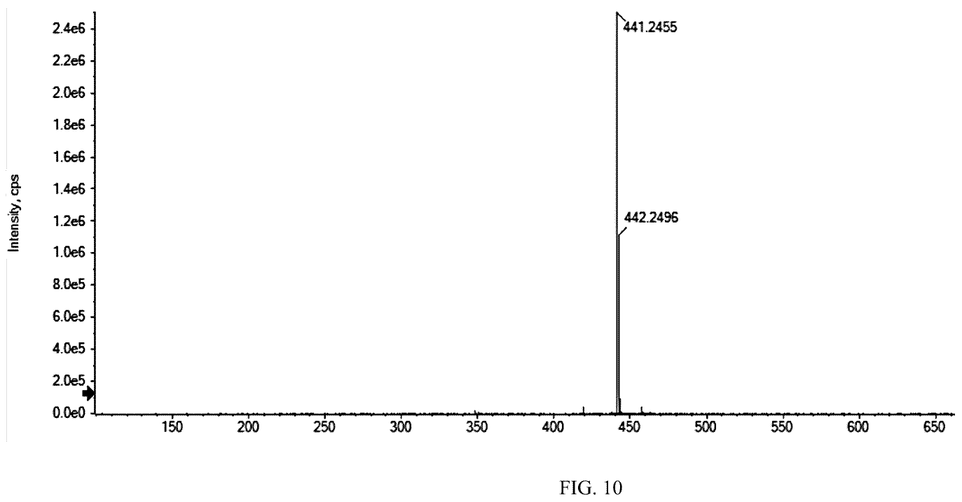

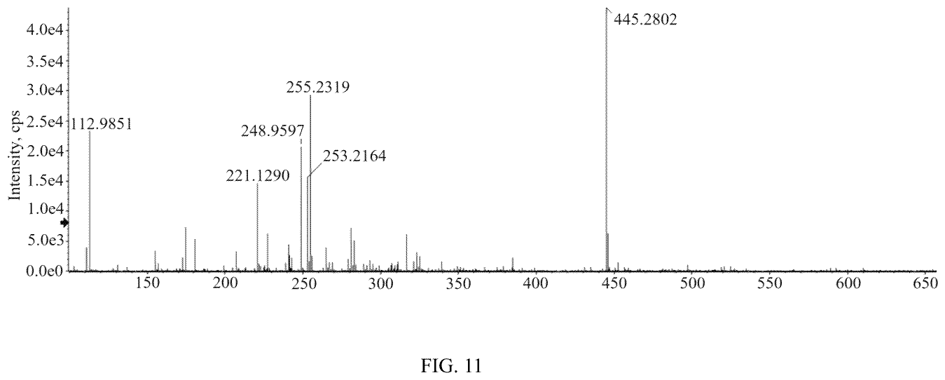

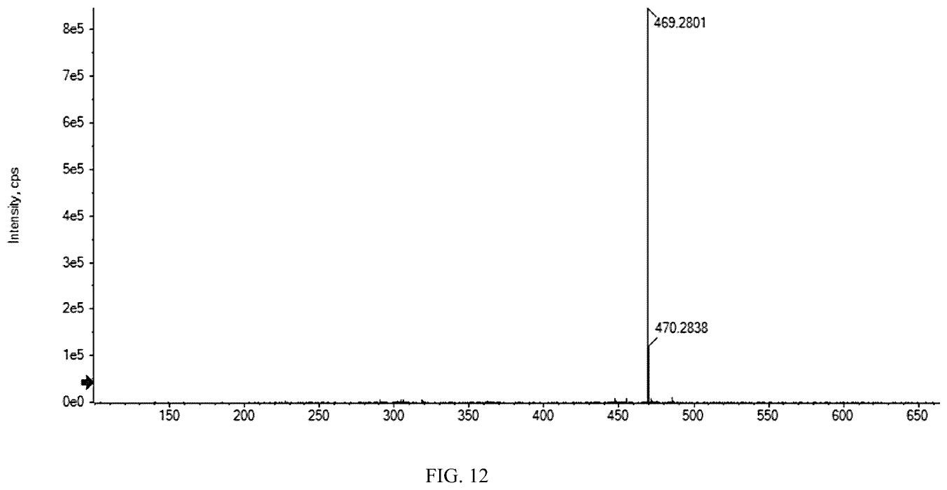

Electrospray ionization-mass spectrometry (ESI-MS) detection is performed by using a mass spectrometer (LCQ Fleet, Thermo): a negative ion ESI-MS spectrum of the compound I is shown in FIG. 7, and a positive ion spectrum of the compound I is shown in FIG. 8; a negative ion ESI-MS spectrum of the compound II is shown in FIG. 9, and a positive ion spectrum of the compound II is shown in FIG. 10; a negative ion ESI-MS spectrum of the compound III is shown in FIG. 11, and a positive ion spectrum of the compound III is shown in FIG. 12.

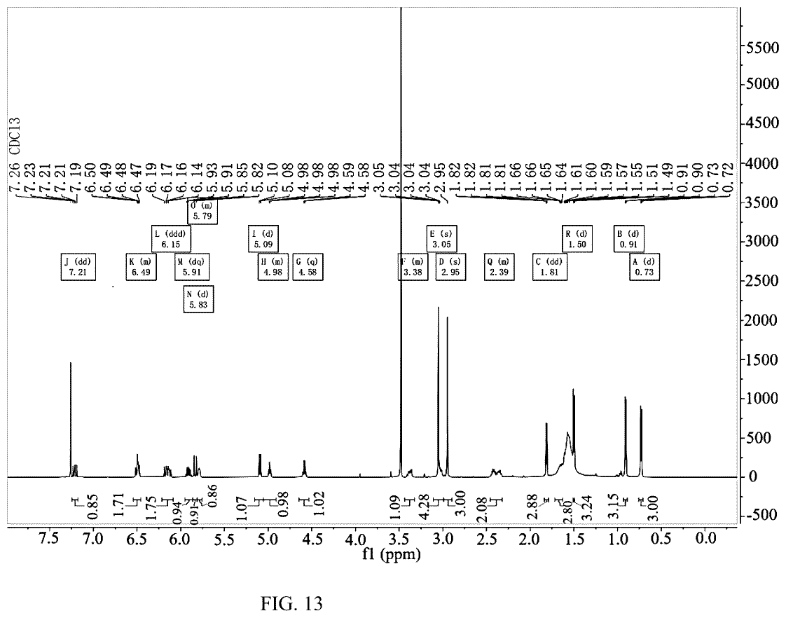

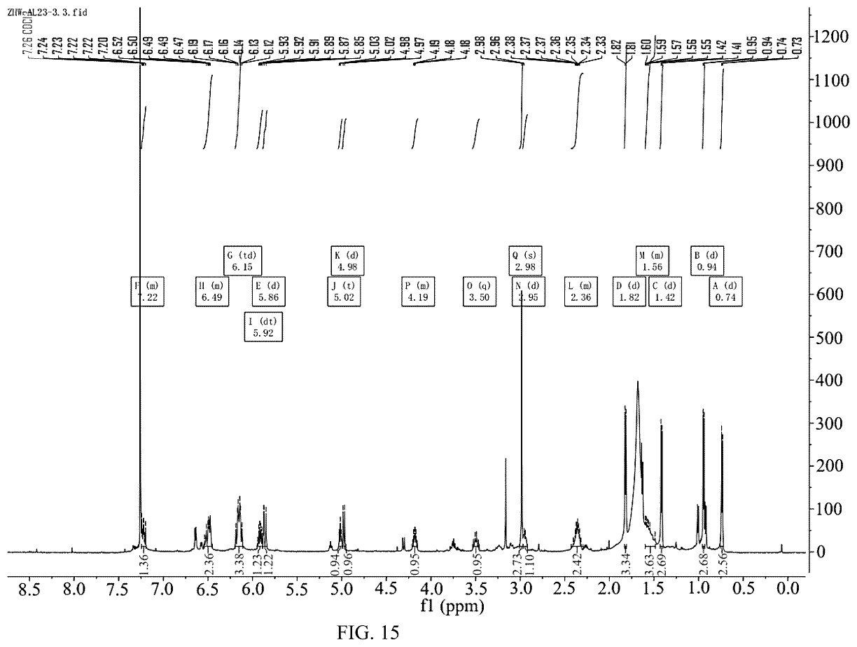

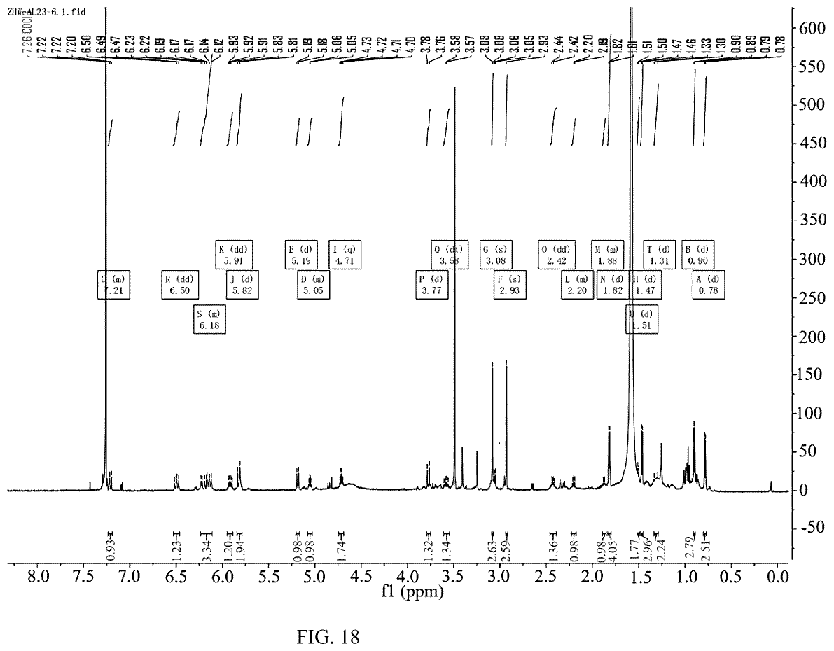

¹H NMR spectra are detected using a nuclear magnetic resonance (NMR) spectrometer (ADVANCE III, Bruker), with results for the compound I, the compound II, and the compound III shown in FIG. 13, FIG. 15, and FIG. 18, respectively.

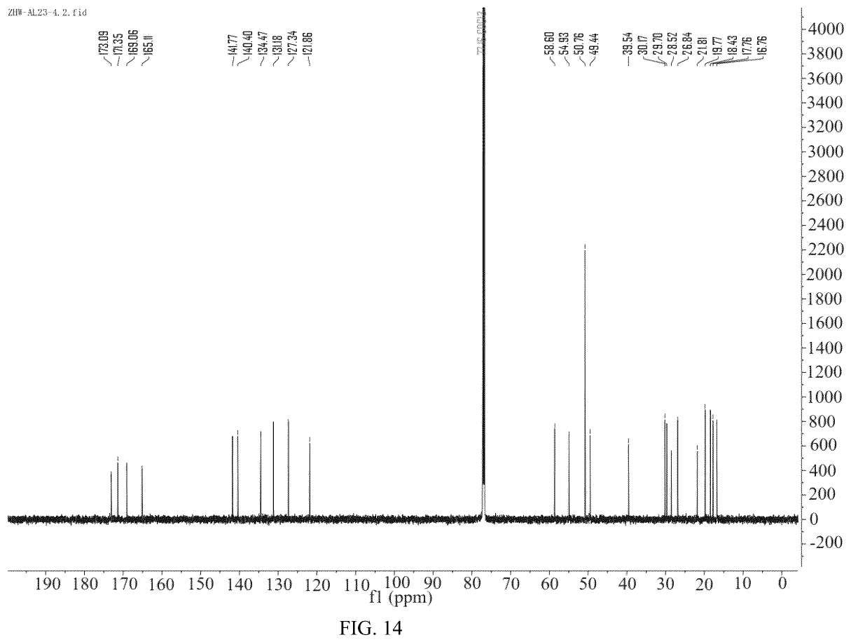

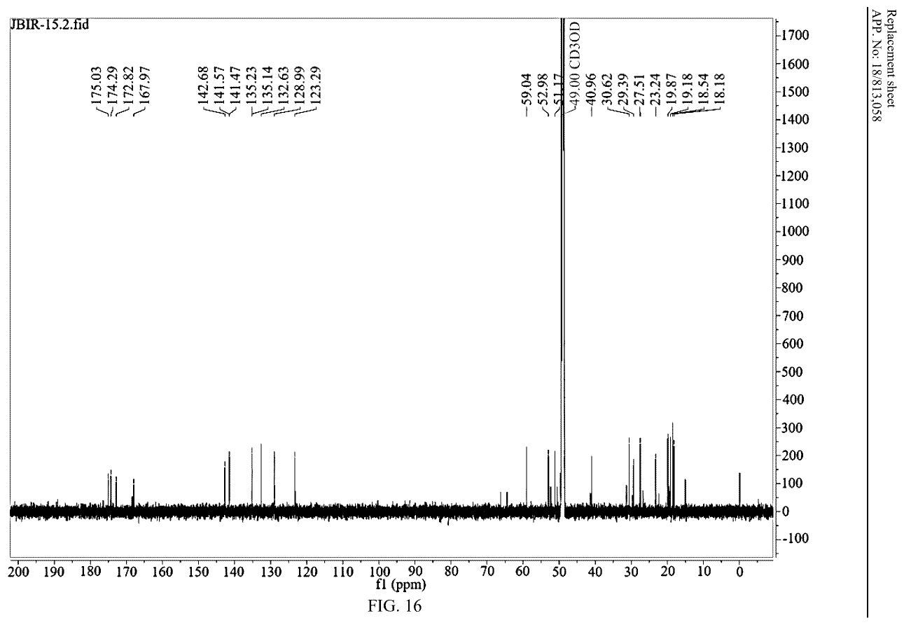

¹³C NMR spectra are detected using the NMR spectrometer (ADVANCE III, Bruker), with results for the compound I and the compound II shown in FIG. 14 and FIG. 16, respectively.

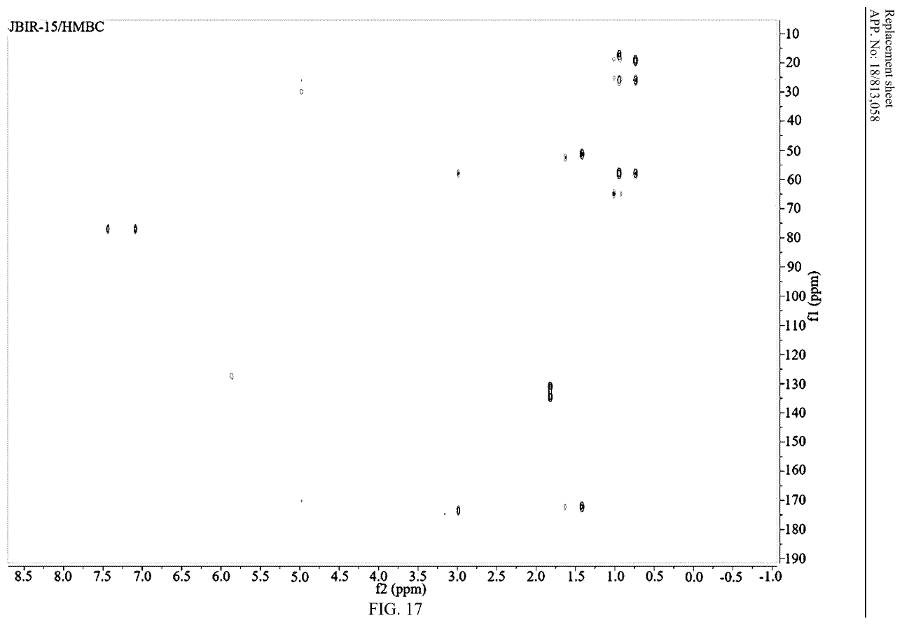

A HMBC spectrum is detected using the NMR spectrometer (ADVANCE III, Bruker), with results for the compound II shown in FIG. 17.

The mass spectrometry data for the compound I are as follows: the ESI-MS spectrum shows molecular ion peaks at m/z 432.2672 ([M]⁺), m/z 431.2638 ([M-H]⁺), and m/z 455.2607 ([M+Na]⁺). By combining the hydrogen and carbon spectrum data, a molecular weight of the compound I is determined to be 432, with a molecular formula C₂₃H₃₆N₄O₄. The NMR data assignments are shown in Table 6.

TABLE 6
¹H-NMR (in CDCl₃) and ¹³C-NMR (in CDCl₃) NMR data
and assignments thereof for the compound I.

| Position | δ_{C} | δ_{H} | |
|---|---|---|---|
| Octa-2,4,6-trienamide | | | |
| 1 | 165.1 | | |
| 2 | 121.9 | 5.83 | (d, J = 14.9 Hz, 1H) |
| 3 | 141.8 | 7.22 | (dd, J = 15.0, 11.2 Hz, 1H) |
| 4 | 127.3 | 6.15 | (td, J = 15.2, 10.9 Hz, H) |
| 5 | 140.0 | 6.50 | (m, 1H) |
| 6 | 131.2 | 5.92 | (dt, J = 14.9, 7.1 Hz, 1H) |
| 7 | 134.5 | 5.79 | (m, 1H) |
| 8 | 18.4 | 1.81 | (dd, J = 7.2, 1.6 Hz, 3H) |
| Orn | | | |
| 1′ | 171.4 | | |
| 2′ | 49.4 | 4.58 | (m, 1H) |
| 3′ | 28.5 | 2.46-2.32 | (m, 1H) |
| | | 1.74-1.58 | (m, 1H) |
| 4′ | 21.8 | 1.74-1.58 | (m, 2H) |
| 5′ | 39.5 | 3.44-3.22 | (m, 1H) |
| | | 3.05 | (m, 1H) |
| 2′-NH | | 6.15 | (m, 1H) |
| 5′-NH | | 6.50 | (m, 1H) |
| Ala | | | |
| 1″ | 173.1 | | |
| 2″ | 54.9 | 4.98 | (m, 1H) |
| 3″ | 16.8 | 1.50 | (d, J = 7.1 Hz, 3H) |
| 2″-N-Me | 30.2 | 2.95 | (s, 3H) |
| N-Me Val | | | |
| 1′″ | 169.1 | | |
| 2′″ | 58.6 | 5.09 | (d, J = 10.4 Hz, 1H) |
| 3′″ | 26.8 | 2.46-2.32 | (m, 1H) |
| 4′″ | 19.7 | 0.91 | (d, J = 6.3 Hz, 3H) |
| 5′″ | 17.8 | 0.73 | (d, J = 7.1 Hz, 3H) |
| 2′″-N-Me | 29.7 | 2.95 | (s, 3H) |

In summary, compared to a non-patent literature (Cyclic tripeptides from the halotolerant fungus Aspergillus sclerotiorum PT06-1[J]. Journal of Natural Products, 2010, 73(6): 1133-7), the structural formula of the compound I is determined as follows:

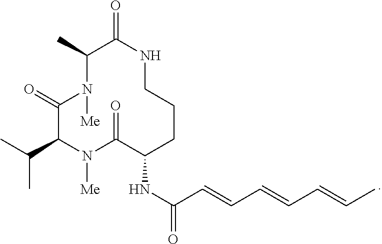 [SMILES: C/C=C/C=C/C=C/C(=O)N[C@H]1CCCNC(=O)[C@H](C)N(C)C(=O)[C@H](C(C)C)N(C)C1=O]

The mass spectrometry data for the compound II are as follows: the ESI-MS spectrum shows molecular ion peaks at m/z 417.2486 ([M-H]⁺) and m/z 441.2455 ([M+Na]⁺). By combining the hydrogen and carbon spectrum data, a molecular weight of the compound II is determined to be 418, with a molecular formula C₂₂H₃₄N₄O₄. The NMR data assignments are shown in Table 7.

TABLE 7
¹H-NMR (in CDCl₃) and ¹³C-NMR (in CDCl₃) NMR data
and assignments thereof for the compound II.

| Position | δ_{C} | δ_{H} | |
|---|---|---|---|
| Octa-2,4,6-trienamide | | | |
| 1 | 168.4 | | |
| 2 | 123.7 | 5.86 | (dd, J = 15.0, 4.2 Hz, 1H) |
| 3 | 143.1 | 7.26-7.18 | (m, 1H) |
| 4 | 129.4 | 6.19-6.12 | (m, 1H) |
| 5 | 141.9 | 6.51-6.46 | (m, 1H) |
| 6 | 133.1 | 6.19-6.12 | (m, 1H) |
| 7 | 135.6 | 5.94-5.89 | (m, 1H) |
| 8 | 19 | 1.82 | (d, J = 6.8 Hz, 3H) |
| Orn | | | |
| 1′ | 175.5 | | |
| 2′ | 51.6 | 5.02 | (t, J = 7.4 Hz, 1H) |
| 3′ | 31.0 | 2.44 -2.31 | (m, 1H) |
| | | 1.60-1.49 | (m, 1H) |
| 4′ | 23.7 | 1.60-1.49 | (m, 2H) |
| 5′ | 41.4 | 2.96-2.94 | (m, 1H) |
| | | 3.52-3.47 | (m, 1H) |
| 2′-NH | | 6.19-6.12 | (m, 1H) |
| 5′-NH | | 6.51-6.46 | (m, 1H) |
| Ala | | | |
| 1″ | 174.7 | | |
| 2″ | 53.4 | 4.22-4.14 | (m, 1H) |
| 3″ | 19.6 | 1.42 | (d, J = 6.9 Hz, 3H) |
| N-Me Val | | | |
| 1′″ | 173.24 | | |
| 2′″ | 59.5 | 4.98 | (d, J = 10.7 Hz, 1H) |
| 3′″ | 28 | 2.44-2.31 | (m, 1H) |
| 4′″ | 20.3 | 0.94 | (d, J = 6.4 Hz, 3H) |
| 5′″ | 18.6 | 0.74 | (d, J = 6.8 Hz, 3H) |
| 2′″-N-Me | 30.0 | 2.98 | (s, 3H) |

In summary, compared to a non-patent literature (JBIR-15, a New aspochracin Derivative, Isolated from a Sponge-Derived Fungus, Aspergillus sclerotiorum Huber Sp080903f04. [J]. Journal of the Agricultural Chemical Society of Japan, 2009, 73(8): 1898-1900.), the structural formula of the compound II is determined as follows:

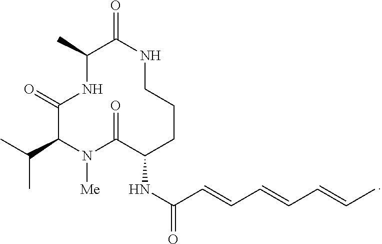 [SMILES: C/C=C/C=C/C=C/C(=O)N[C@H]1CCCNC(=O)[C@H](C)NC(=O)[C@H](C(C)C)N(C)C1=O]

The mass spectrometry data for the compound III are as follows: The ESI-MS spectrum shows molecular ion peaks at m/z 445.2802 ([M-H]⁺) and m/z 469.2801 ([M+Na]⁺). By combining the hydrogen and carbon spectrum data, a molecular weight of the compound III is determined to be 446, with a molecular formula C₂₄H₃₈N₄O₄. The NMR data assignments are shown in Table 8.

TABLE 8
¹H-NMR (in CDCl₃) NMR data and assignments
thereof for the compound III.

| Position | δ_{H} | |
|---|---|---|
| Octa-2,4,6-trienamide | | |
| 1 | | |
| 2 | 5.82 | (d, J = 14.7 Hz, 1H) |
| 3 | 7.21 | (m, 1H) |
| 4 | 6.18 | (m, 1H) |
| 5 | 6.50 | (m, 1H) |
| 6 | 6.91 | (m, 1H) |
| 7 | 5.82 | (m, 1H) |
| 8 | 1.82 | (d, J = 6.8 Hz, 3H) |
| Orn | | |
| 1′ | | |
| 2′ | 4.17 | (m, 1H) |
| 3′ | 1.81 | (m, 1H) |
| | 1.88 | (m, 1H) |
| 4′ | 1.35-1.30 | (m, 2H) |
| 5′ | 1.51 | (m, 2H) |
| 6′ | 3.68 | (m, 1H) |
| | 3.76 | (m, 1H) |
| 2′-NH | 6.18 | (m, 1H) |
| 6′-NH | 6.18 | (m, 1H) |
| Ala | | |
| 1″ | | |
| 2″ | 5.08-5.03 | (m, 1H) |
| 3″ | 1.47 | (d, J = 6.9 Hz, 3H) |
| 2″-N-Me | 3.08 | (s, 3H) |
| N-Me Val | | |
| 1′″ | | |
| 2′″ | 5.19 | (d, J = 10.5 Hz, 1H) |
| 3′″ | 2.42 | (m, 1H) |
| 4′″ | 0.90 | (d, J = 6.4 Hz, 3H) |
| 5′″ | 0.78 | (d, J = 6.8 Hz, 3H) |
| 2′″-N-Me | 2.93 | (s, 3H) |

In summary, compared to a non-patent literature (Cyclic tripeptides from the halotolerant fungus Aspergillus sclerotiorum PT06-1[J]. Journal of Natural Products, 2010, 73(6): 1133-7), the structural formula of the compound III is determined as follows:

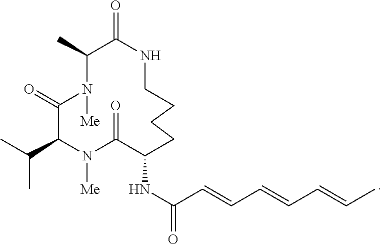 [SMILES: C/C=C/C=C/C=C/C(=O)N[C@H]1CCCCNC(=O)[C@H](C)N(C)C(=O)[C@H](C(C)C)N(C)C1=O]

### Embodiment 4: Activity Detection

1. Antifungal Activity Against Candida albicans

Candida albicans ATCC 10231, as an indicator strain, is activated for 3 days at 30° C. on the Sabouraud solid medium, and then amplified for 2 days at 30° C. and 180 rpm in the Sabouraud liquid medium to obtain an indicator liquid for antifungal activity testing.

An outermost ring of a 96-well plate is filled with 100 μL of the Sabouraud liquid medium for blank culture to prevent contamination; a first well of the 96-well plate is added with samples and the indicator liquid, with a total solution volume of 200 μL and a sample concentration of 128 μg/mL in the first well. A “serial dilution method” is used to achieve sample concentrations of 64, 32, 16, 8, 4, 2, 1, 0.5, and 0.25 μg/mL respectively in subsequent wells. Each sample has three parallel controls, and is incubated for 48 hours at 30° C. Then, the absorbance of the indicator liquid is measured at 600 nm using a microplate reader, and minimum inhibitory concentrations (MIC) of the compounds are recorded. The samples are the compound I, the compound II, and the compound III, with Amphotericin B as the control.

Results show that the MIC value of the compound II against the Candida albicans ATCC 10231 is 32 μg/mL.

TABLE 9
Antifungal activity against Candida albicans test results
of the compound I, the compound II, and the compound III

| Compound | I | II | III | Amphotericin B |
|---|---|---|---|---|
| MIC value against the Candida | >128 | 32 | >128 | 2 |
| albicans ATCC 10231 (μg/mL) | | | | |

### 2. 2,2-Diphenyl-1-Picrylhydrazyl (DPPH) Antioxidant Activity Test

1 mg of solid DPPH is dissolved in 24 mL of methanol and then ultrasonicated for 5 min, followed by shaking thoroughly to obtain DPPH solution to ensure uniformity throughout the DPPH solution. 1 mL of the DPPH solution is taken and diluted by adding 0.5 mL of 95% methanol to obtain a diluted solution with an absorbance in a range of 0.6-1.0. The samples are added with methanol to prepare 1 mg/mL of sample solutions. The sample solutions are separately added to the DPPH solution while mixing continuously with a small start amount to obtain mixed solutions and an addition amount of the sample solutions gradually increases. Solution decolorization is observed when adding the sample solutions. When color of the mixed solutions has essentially faded, a maximum amount of the added sample solution is recorded. Based on the maximum amount, five additional amounts are set in an arithmetic sequence moving backwards. A final measurement is performed according to the arithmetic sequence, and for each amount, three parallel data points are measured. An absorbance value (A value) at each amount may be measured after placing the mixed solutions in a 37° C. oven for half an hour. An experimental antioxidant concentration serves as an x-axis and a scavenging rate serves as a y-axis, then a linear regression equation is calculated. 50% scavenging rate is substituted into the linear regression equation to obtain a corresponding x-axis value, which is a half-maximal inhibitory concentration (IC₅₀) value. The samples are the compound I, the compound II, and the compound III, with vitamin C (Vc) as the control.

The scavenging rate=((A₀−A)/A₀)×100; where A₀ represents a value before adding the sample solutions, and A represents a value after adding the sample solutions.

Results show that the IC₅₀ values of the compound I, the compound II, and the compound III are 420 μg/mL, 400 μg/mL, and 430 μg/mL respectively.

TABLE 10
Antioxidant activity test results of the compound
I, the compound II, and the compound III

| Compound | I | II | III | Vc |
|---|---|---|---|---|
| Antioxidant activity | 420 | 400 | 430 | 9 |
| IC₅₀ value (μg/mL) | | | | |

### 3. Anti-Tumor Activity Test

A Sulforhodamine B (SRB) colorimetric method is used to perform in vitro growth inhibition experiments on tumor cells with the isolated monomeric compounds

### (1) Tumor Cells

A human brain glioma cell line (HEB) and a human hepatocellular carcinoma cell line (Hep-G2) both from Cell Resource Center, Institute of Basic Medical Sciences (CAMS/PUMC), China.

Tumor cells in a logarithmic growth phase are selected and digested with trypsin followed by adjusting a cell concentration to 2×10⁴ cells/mL with a Roswell Park memorial institute 1640 (RPMI 1640) medium containing 10% fetal bovine serum to obtain a tumor cell suspension.

### (2) Drugs and Reagents

0.4% SRB solution: 0.8 g of SRB is weighed and then dissolved in 200 mL of 1% acetic acid aqueous solution to obtain the 0.4% SRB solution, stored at room temperature.

50% trichloroacetic acid (TCA) solution: 50 g of TCA is weighed and added with water to make up to 100 mL to obtain the 50% TCA solution, stored at 4° C.

10 millimoles (mM) Tris(hydroxymethyl)aminomethane (Tris-based) solution: 0.6057 g of Tris-base is weighed and then added with water to make up to 500 mL followed by adjusting pH to 10.5 to obtain the 10 mM Tris-based solution, stored at 4° C.

Sample solutions: the compound I, the compound II, and the compound III prepared in the embodiment 3 are separately added with DMSO to prepare 200 μM of the sample solutions.

Control antibiotic solution: The antibiotic 5-fluorouracil (5-FU) is diluted with DMSO to prepare 100 μM of the control antibiotic solution.

### (3) Tumor Cell Viability Test

The tumor cell suspensions in Table 11 are inoculated into a 96-well culture plate at 190 μL per well, and then incubated at 37° C. with 5% carbon dioxide (CO₂) for 24 hours. The culture wells are divided into drug wells, control wells, and blank wells.

The drug wells are added with 10 μL of the sample solutions respectively to achieve a final drug concentration of 10 μM. The control wells are added with 10 μL of the control antibiotic solution to achieve a final drug concentration of 5 μM. The blank wells are added with 10 μL of the RPMI 1640 medium containing 10% fetal bovine serum and an equal volume of DMSO as a solvent. The 96-well culture plate is then at 37° C. with 5% CO₂ for incubation for 3 days followed by removing culture medium. Then, each well of the 96-well culture plate is slightly added with 100 μL of the 50% TCA solution pre-cooled at 4° C., and then allowed to stand for 5 min, followed by moving the 96-well culture plate to 4° C. for 1 hour to fix cells to obtain a cell-fixed plate. After discarding the fixing solution from the cell-fixed plate, the wells of the cell-fixed plate are washed five times with distilled water to remove TCA and then air-dried for 1 hour to obtain a first dried plate. Each well of the first dried plate is added with 80 μL of the 0.4% SRB solution for staining at room temperature for 30 min to obtain a stained plate. After discarding the staining solution of the stained plate, the stained plate is washed five times by 1% acetic acid aqueous solution to fully remove unbound SRB and then air-dried for 1 hour to obtain a second dried plate. Each well of the second dried plate is added with 150 μL of the 10 mM Tris-based solution for dissolving, and then oscillated for 5 min on a mini shaker (Mini shaker Kylin-Bell Lab instruments) to obtain an oscillated plate. Samples from each well of the oscillated plate are taken and an OD value at 570 nm is measured for each sample by using an M5 microplate reader. A tumor cell growth inhibition rate is calculated according to a formula, and the results are shown in Table 11.

$$The tumor cell growth inhibition rate ( \% ) = ( O {D}_{control} - O {D}_{drug} ) / ( {OD}_{control} - O {D}_{blank} ) \times 100 \% .$$

The results show that at a concentration of 10 μM, the JBIR-15 and the sclerotiotide C have inhibition rates of 11.78% and 21.09%, respectively, on the human hepatocellular carcinoma cell line Hep-G2

TABLE 11
anti-tumor cell activity test results of the compound
(I), the compound (II), and the compound (III)

| | | human hepatocellular |
|---|---|---|
| | human brain glioma cell line | carcinoma cell line Hep-G2 |
| Compound | HEB growth inhibition rate % | growth inhibition rate % |
| (I) | −19.94% | −17.88% |
| (II) | −6.04% | 11.78% |
| (III) | −13.69% | 21.09% |
| Positive | 69.55% | 71.32% |
| control 5-FU | | |

The aforementioned is merely specific embodiments of the disclosure, but the scope of protection of the disclosure is not limited thereto. Any variations or substitutions that would occur to those skilled in the art without creative effort should be covered within the scope of protection of the disclosure. Therefore, the scope of protection of the disclosure should be determined by the claims.

What is claimed is:

1. An Aspergillus sp. L14-OE::laeA2 capable of producing cyclic tripeptides, preserved at China Center for Type Culture Collection (CCTCC) on Oct. 27, 2023, wherein a preservation number is CCTCC NO: M20232057 and a preservation address is Wuhan University, Wuhan, China.

2. An application method comprising: applying the Aspergillus sp. L14-OE::laeA2 as claimed in claim 1 in producing the cyclic tripeptides.

3. The application method as claimed in claim 2, specifically comprising: fermenting and culturing the Aspergillus sp. L14-OE::laeA2 to obtain fermentation broth, and separating and purifying the fermentation broth to obtain the cyclic tripeptide compounds; wherein the cyclic tripeptides comprise compound (I), compound (II) and compound (III) presented as follows;

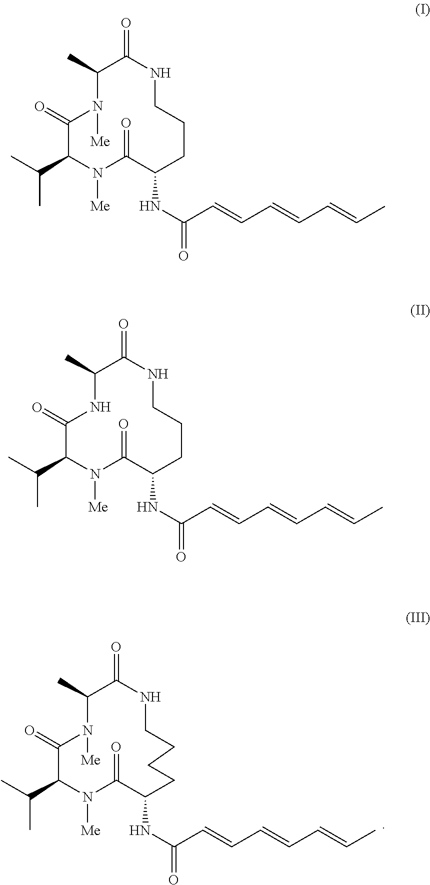

4. The application method as claimed in claim 3, wherein the fermenting and culturing the Aspergillus sp. L14-OE::laeA2 comprises:
(1) inoculating the Aspergillus sp. L14-OE::laeA2 into a potato dextrose agar (PDA) medium and then activating the Aspergillus sp. L14-OE::laeA2 at 30° C. for 3-4 days to obtain activated Aspergillus, inoculating the activated Aspergillus into a potato dextrose broth (PDB) medium and then culturing the activated Aspergillus at 30° C. and 180-200 revolutions per minute (rpm) for 3 days to obtain seed liquid; and
(2) inoculating the seed liquid in the step (1) into a Czapek medium at a volume concentration in a range of 1-5% followed by culturing at 30° C. and 180-200 rpm for 15 days to obtain the fermentation broth.

5. The application method as claimed in claim 4, wherein the Czapek medium comprises 30 grams per liter (g/L) of sucrose, 3 g/L of sodium nitrate, 0.5 g/L of magnesium sulfate heptahydrate, 0.5 g/L of potassium chloride, 0.01 g/L of ferrous sulfate, and 1 g/L of dipotassium hydrogenphosphate, with water as solvent and potential of hydrogen (pH) of natural.

6. The application method as claimed in claim 3, wherein the separating and purifying the fermentation broth comprises:
filtering the fermentation broth through eight-layer gauze to separate fungal liquid and mycelium, adding the fungal liquid with an equal volume of ethyl acetate followed by extracting to obtain an upper layer, and concentrating the upper layer to dryness by rotary evaporation under reduced pressure to obtain a crude fermentation extract; and dissolving the crude fermentation extract with methanol alcohol followed by diluting with the methanol alcohol to obtain a diluted solution, filtering the diluted solution with a 0.22 micrometers (μm) organic filter to obtain a filtrate, performing isocratic elution on the filtrate by an analytical column of high-performance liquid chromatography (HPLC) with a mobile phase of acetonitrile:water at a volume ratio of 35:65 to obtain effluents from 6^{th} minute (min) to 7^{th} min, 8^{th} min to 9^{th} min and 10.8^{th} min to 11.2^{nd} min respectively, and removing solvents from the effluents by rotary evaporation followed by drying at 25° C. to obtain the compound (I), the compound (II) and the compound (III) respectively.

7. The application method as claimed in claim 6, wherein HPLC conditions comprise: an ultraviolet-visible (UV-Vis) liquid chromatography instrument, a detector, and a HPLC pump; the analytical column is a C18 column of 4.6×250 millimeters (mm), a flow rate is 1.0 milliliters per minute (mL/min), a column temperature is 40° C., a detection wavelength is 210 nanometers (nm), and an injection volume is 10 microliters (μL).

8. An application method comprising: applying the cyclic tripeptides produced by strain Aspergillus sp. L14-OE::laeA2 as claimed in claim 1 in preparing antioxidants drugs.

9. An application method comprising: applying the cyclic tripeptides prepared by strain Aspergillus sp. L14-OE::laeA2 as claimed in claim 1 in preparing drugs to inhibit activity of tumor cells.

10. The application method as claimed in claim 9, wherein the tumor cells comprise: a human brain glioma cell line (HEB) and a human hepatocellular carcinoma cell line (Hep-G2).
